(19) United States
(12) Patent Application Publication
(10) Pub. No.: US 2024/0238779 A1
(43) Pub. Date: Jul. 18, 2024

(54) DIAGNOSTIC TESTING

(71) Applicant: DX TEK LTD, London (GB)

(72) Inventor: Gordon Jowett, London (GB)

(21) Appl. No.: 18/561,792

(22) PCT Filed: May 17, 2022
(86) PCT No.: PCT/GB2022/051230

§ 371 Date: Nov. 17, 2023

(30) Foreign Application Priority Data
May 18, 2021 (GB) 2107090.9

(51) Int. Cl. B01L 3/00; G01N 21/84; G16H 40/67

(52) U.S. Cl. CPC B01L 3/5023; G01N 21/8483; G16H 40/67; B01L 2200/0689; B01L 2200/16; B01L 2300/023; B01L 2300/0825; B01L 2300/123

(57) ABSTRACT

A diagnostic test device is described. The device comprises a sample-processing section (4) comprising a test area (9; FIG. 5) containing a test reagent and a fluid conveyor (91; FIG. 5) for receiving a liquid sample (6) and transferring the liquid sample to the test area. The device comprises an electronic section (12) comprising a measurement section (5) for measuring properties in or of the test area, an antenna (17), a modulator-demodulator (19) coupled to the antenna, a data processing unit (14) coupled to the measurement section and to the modulator-demodulator and an energy-harvesting unit coupled to the antenna and to the data processing unit. The data processing unit is configured, upon receiving a measurement from the measurement section, to generate a message containing the measurement and/or a result of processing the measurement and to transmit the message via the modulator-demodulator and the antenna. The device comprises a frame (31; FIG. 5), a chassis (51; FIG. 5) coupled to the frame and slidably moveable with respect to the frame, using a slider (59; FIG. 5), between first and second positions, wherein the chassis carries the test area. In the second position, the sample is transferable to the test area or the sample is receivable by the fluid conveyor. The device comprises a wrapper (i11; FIG. 5) comprising a flexible substrate (13). The electronic section (12) is supported on the substrate, wherein the wrapper encloses the frame and chassis, and wherein the wrapper includes a first aperture (113) for allowing the fluid conveyor to receive the sample and a second aperture (114; FIG. 5) for allowing a user to access to the slider.

### FIELD

The present invention relates to diagnostic testing, particularly, but not exclusively, immunoassay diagnostic testing.

### BACKGROUND

Diagnostic test system for detecting the presence or amount of a substance, such as blood sugar, protein, antigen or other molecule, are becoming increasingly common and are being developed for use at point-of-care and in the home. Well known examples include the COVID-19 antigen test and the Clearblue® pregnancy test.

Some diagnostic test systems, such as the COVID-19 antigen test, are simple and consist of, for instance, an immunoassay lateral flow device. These types of device tend to be simple and cheap, but specific. They are, however, reliant on visual interpretation, and only provide a binary result and not a quantitative result. Furthermore, these types of device rely on the user to scan in a bar code to provide traceability. Traceability is very useful from an epidemiological stand point in situations such as a pandemic.

Other diagnostic test systems are more complex and consist of a consumable, which often comprise immunoassay lateral flow device, or another type of microfluidic cartridge, for example with an immunoassay based on electrochemical detection and a reader/analyser. It may be possible to use the same reader/analyser to test for different substances. However, this type of system is more expensive and particularly when the reader/analyser is to be supplied as an OEM item, often the consumable and/or the reader/analyser have to be adapted to allow them to work together. This is a significant challenge particularly when developing a platform system, where a reader/analyser is being developed to run a potentially extensive menu of different tests. For example, the different tests may have different configurations (e.g., different arrangement and/or numbers of electrodes, or liquid buffers), requiring different interfaces between the reader/analyser and the consumable which may not have been envisaged when designing the reader.

Some diagnostic systems use a smartphone, in particular, taking advantage of the smartphone camera to image a microfluidic system.

### SUMMARY

According to a first aspect of the present invention there is provided a diagnostic test device. The device comprises a sample-processing section comprising a test area containing a test reagent and a fluid conveyor for receiving a liquid sample and transferring the liquid sample to the test area. The device comprises an electronic section comprising a measurement section for measuring properties in or of the test area, an antenna, a modulator-demodulator coupled to the antenna, a data processing unit (or “logic”) coupled to the measurement section and to the modulator-demodulator and an energy-harvesting unit coupled to the antenna and to the data processing unit. The data processing unit is configured, upon receiving a measurement from the measurement section, to generate a message containing the measurement and/or a result of processing the measurement and to transmit the message via the modulator-demodulator and the antenna. The device comprises a frame, a chassis or sliding element coupled to the frame and slidably moveable with respect to the frame, using a slider, between first and second positions. The chassis may carry the test area. In the second position, the sample is transferable to the test area or the sample is receivable by the fluid conveyor. The device comprises a wrapper comprising a flexible substrate. The electronic section is supported on the substrate, wherein the wrapper encloses the frame and chassis, and wherein the wrapper includes a first aperture for allowing the fluid conveyor to receive the sample and a second aperture for allowing a user to access to the slider and at least one removeable sticker arranged to cover the first and second apertures.

The device may comprise at least one lateral flow strip, each strip providing a respective test area. The device may comprise two, three or four lateral flow strips.

The measurement section may comprise at least one light source and at least one light/image sensor. The at least one light source and the at least one light/image sensor may be arranged to measure transmission, absorbance, and/or reflectance through or by the test area. Each test area can be provided with at least one pair comprising a light source and a light/image sensor.

The measurement section may comprise at least one electrochemical sensor. The electrochemical sensor may comprise an electrode or wire coated with a receptor which binds to a specific target molecule.

The first aperture may be aligned with the fluid conveyor such that, in the first position, the sample is receivable by the fluid conveyor and, in the second position, the test area is in fluid communication with the fluid conveyor.

The device may further comprise a burstable buffer capsule arranged such that moving chassis between the first and second positions cause the burstable buffer capsule to burst and release the buffer onto conveyor pad. The burstable buffer capsule may be held in a chamber in the frame and the chassis may comprise a projecting member arranged to enter into the chamber when the chassis is moved into the second position.

The chassis may carry the fluid conveyor and the test area may be in permanent fluid communication with the fluid conveyor, such that, in the first position, the fluid conveyor is stowed inside the wrapper, and, in the second position, the fluid conveyor is deployed such that the sample is receivable by the fluid conveyor.

The wrapper and the at least one removeable sticker are preferably arranged to provide a gas-tight enclosure. The device may further comprise a colour-changing desiccant indicator and the wrapper may include a transparent window positioned such that the colour-changing desiccant indicator is visible.

The device is preferably battery-less.

The chassis or sliding element may deploy and retract sample taking element(s). The chassis or sliding element may be arranged for reciprocal movement. The chassis or sliding element may be arranged to burst a buffer capsule containing a buffer and dispense the buffer at a given point in the assay process. The chassis or sliding element may be configured to bring sample collection and conditioning elements into contact with a sample test section at a given point during the test. The device may not be lateral flow device

According to a second aspect of the present invention there is provided a device for providing an interface to a diagnostic test device. The device comprises a controller, a short-range wireless communication module (such as NFC), a user interface (such as a display and/or a speaker) and a user input device (such as a touch screen and/or microphone) for receiving a voice command. The controller is configured to provide instructions to a user, via the user interface, to perform a test using the diagnostic test device, to receive an input from the user, via the user input device, to start a timer, to determine if a given period of time has passed and, upon a positive determination, to activate the short-range wireless communication module so as to provide power to the diagnostic test device, to receive a signal from the diagnostic test device and to present the result to the user, via the user interface.

According to a third aspect of the present invention there is provided a diagnostic test system. The system comprises the diagnostic test device of the first aspect and the interface device of the second aspect and/or a hand-held communications device (such as smart phone or tablet computer) capable of short-range wireless communication with the diagnostic test device. The system may further comprise a remote server.

According to a fourth aspect of the present invention there is provided a method, comprising, upon receiving power from an energy-harvesting module a first time transmitting data stored in non-volatile memory via a short-range communication module, upon receiving power from the energy-harvesting module a second, later time retrieving instructions from the non-volatile memory, performing a measurement using a measuring section and processing measurement data from the measuring section in dependence upon the instructions and transmitting measurement data and/or a result obtained by processing the measurement data via the short-range communication module.

The data may comprise a test type and/or an expiry data, instructions to be presented to a user, and/or calibration data.

According to a fifth aspect of the present invention there is provided logic arranged to perform the method of the fourth aspect.

According to a sixth aspect of the present invention there is provided a method comprising causing transmission of a short-range wireless communication signal to a testing device for providing the testing device with power, receiving data wirelessly from the testing device, providing instructions to a user, via a user interface (such as a display and/or a speaker), to perform a test using the diagnostic test device in dependence upon the data received from the testing device, receiving an input from the user, via a user input device (such as a touch screen and/or microphone), to start a timer, determining if a given period of time has passed, an, upon a positive determination, causing transmission of a further short-range wireless communication signal to the testing device for providing the testing device with power receiving a signal from the diagnostic test device, the signal containing measurement data and/or result and to present the measurement and/or result to the user via the user interface.

The period may be specified in the data received from the testing device.

According to a seventh aspect of the present invention there is provided a computer program comprising instructions for performing the method of the sixth aspect.

According to an eight aspect of the present invention there is provided a computer program product comprising a computer-readable medium (which is may be non-transitory) storing thereon the computer program of the seventh aspect.

### BRIEF DESCRIPTION OF THE DRAWINGS

Certain embodiments of the present invention will now be described, by way of example, with reference to the accompanying drawings, in which:

FIG. 1 is schematic block diagram of a testing system comprising a testing device and an interface device;

FIG. 2 illustrates an example of a fluid processing section and an example of a measuring section in a testing device;

FIG. 3 is a perspective view of a well-type testing device without its label;

FIG. 4 is a perspective view of a well-type testing device with its label and peel-off sticker;

FIG. 5 is a perspective, exploded view of a well-type testing device;

FIG. 6 is a perspective view of the back of well-type testing device shown in FIG. 3 without its label;

FIG. 7 is plan view of circuitry incorporated into a label of a well-type testing device;

FIG. 8 is perspective view of circuitry incorporated into a label of a testing device;

FIG. 9A is a cross-section view of a well-type testing device taken along a line X-X′ in FIG. 3 when the testing device is in a first state;

FIG. 9B is a cross-section view of a well-type testing device taken along a line X-X′ in FIG. 3 when the testing device is in a second state;

FIG. 10A is a cross-section view of a well-type testing device taken along a line Y-Y′ in FIG. 3 when the testing device is in a first state;

FIG. 10B is a cross-section view of a well-type testing device taken along a line Y-Y′ in FIG. 3 when the testing device is in a second state;

FIG. 11A is a plan view of a well-type testing device when the testing device is in a first state;

FIG. 11B is a plan view of a well-type testing device when the testing device is in a second state;

FIG. 11C is a plan view of a well-type testing device when the testing device is returned to a first state after having been in a second state;

FIG. 12 is a perspective view of a tab-based testing device with its label and peel-off sticker;

FIG. 13 is plan view of circuitry incorporated into a label of a tab-based testing device;

FIG. 14 is a perspective, exploded view of a tab-type testing device;

FIG. 15 is a perspective view of the back of tab-type testing device shown in FIG. 12 without its label;

FIG. 16A is a perspective view of a well-type testing device without its label in a first state;

FIG. 16B is a perspective view of a well-type testing device without its label in a second state;

FIG. 17A is a plan view of a tab-based testing device when the testing device is in a first state;

FIG. 17B is a plan view of a tab-based testing device when the testing device is in a second state;

FIG. 17C is a plan view of a tab-based testing device when the testing device is so returned to a first state after having been in a second state;

FIG. 18A is a plan view of another well-type testing device when the testing device is in a first state;

FIG. 18B is a plan view of another well-type testing device when the testing device is in a second state;

FIG. 19 is a process flow diagram of a method of testing;

FIG. 20 illustrates data stored in non-volatile memory in a testing device;

FIG. 22 is a schematic block diagram of a home testing system;

FIG. 22 is a schematic block diagram of a professional testing system; and

FIG. 23 illustrates another example of a fluid processing section.

### DETAILED DESCRIPTION OF CERTAIN EMBODIMENTS

In the following description, like parts are denoted by like reference numerals.

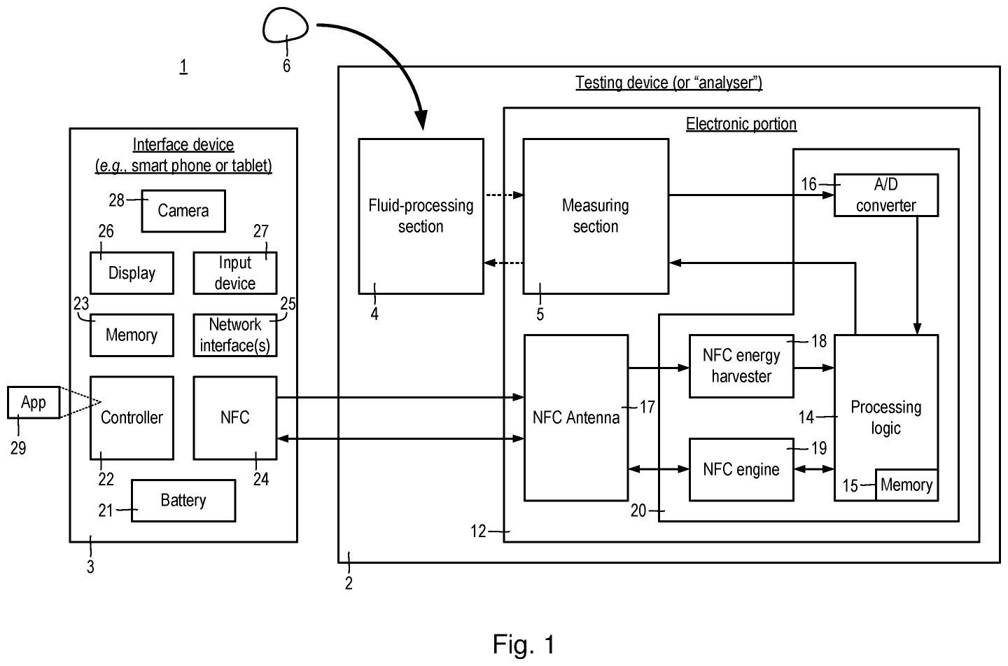

Referring to FIG. 1, a testing system 1 is shown which comprises a testing device 2 (or “assay device”) and an interface device 3 for interfacing with the testing device 2.

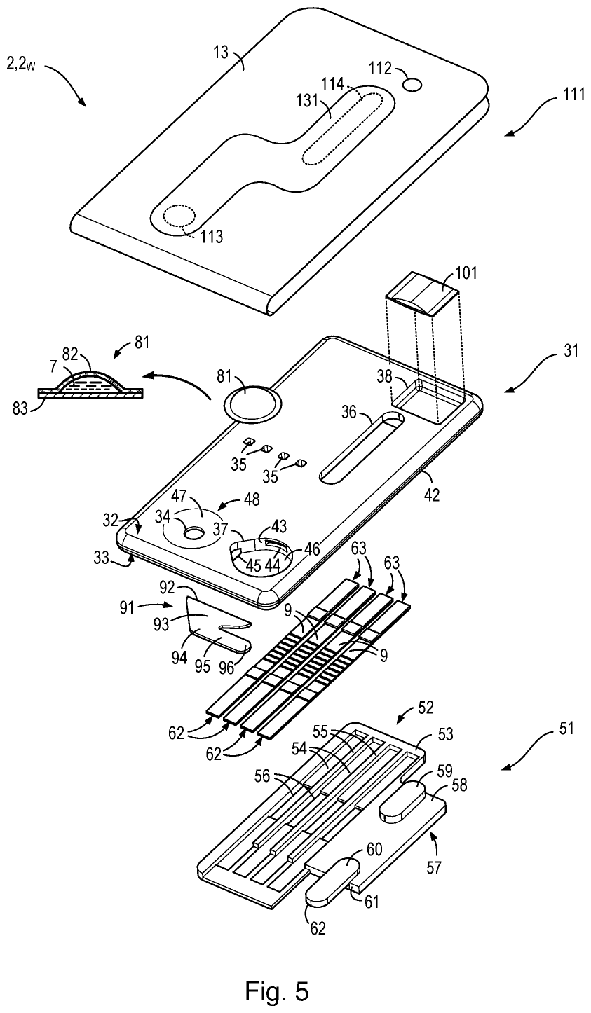

The testing device 2 comprises a fluid processing section 4 and a measuring section 5. The fluid processing section 4 generally receives a sample 6 to be tested and can process the sample, for example, by metering the sample, filtering the sample and/or by separating the sample into different components, and optionally combine the sample (or a portion thereof) with a buffer 7 (FIG. 5) and combine the sample (or portion thereof) with one or more reagents (not shown) which may be dry or wet. The measuring section 5 generally takes measurements, for example, optically, electrically, or via another suitable approach.

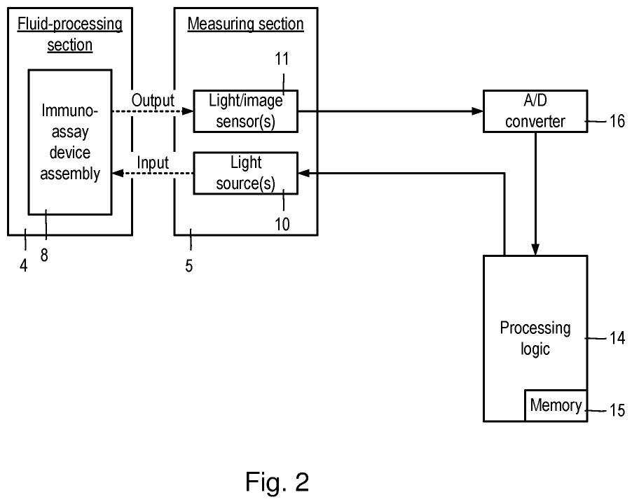

Referring also to FIG. 2, the fluid processing section 4 takes the form of an immunoassay device assembly 8 comprising an array of four lateral flow immunoassay devices 9 (FIG. 5). Other forms of immunoassay device can be used and there may be fewer immunoassay devices 9, e.g., one, two or three, or more immunoassay devices 9, such as five, six or more. The measuring section 5 takes the form of a spectrometer comprising one or more light sources 10, such as an array of light emitting diodes (LEDs), and one or more corresponding light sensors 11, such as an array of photodiodes (PDs). A source-sensor pair 10, 11 may be arranged to measure transmission, absorbance, and/or reflectance through or by portion(s) of immunoassay so device 9.

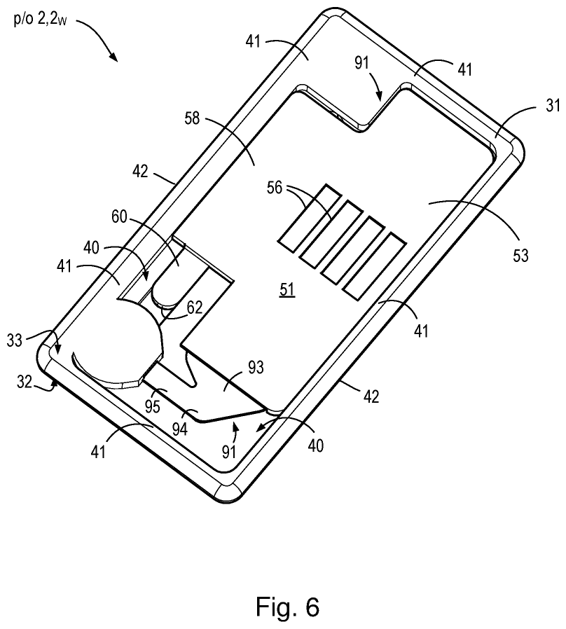

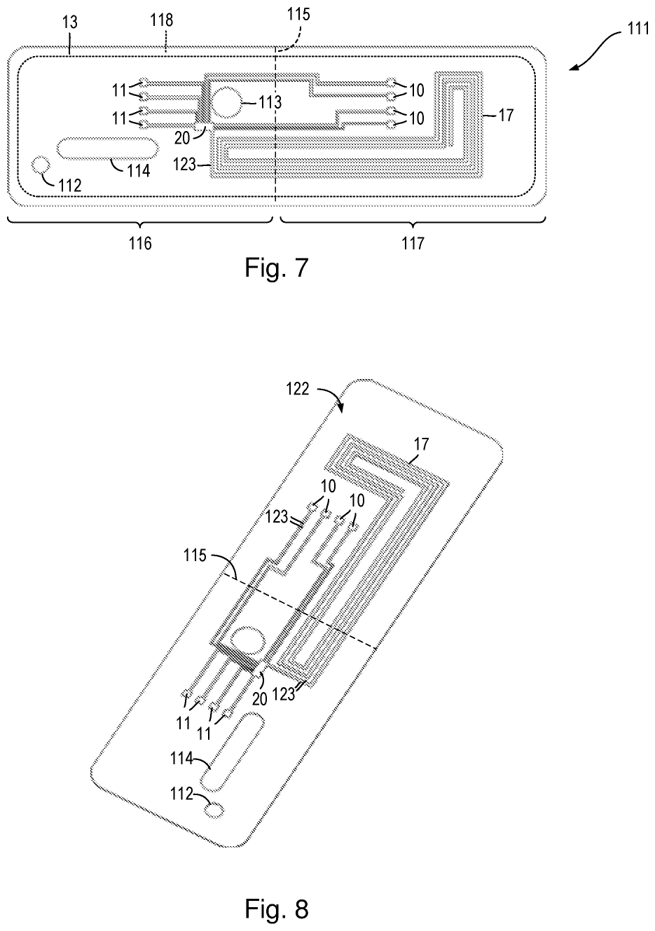

Referring again to FIG. 1, the testing device 2 also comprises an electronic portion 12 (or “circuitry”) supported on a flexible substrate 13 (FIG. 7), for example, formed from polyethylene (PE), polyethylene terephthalate (PET) or other suitable material, which serves as part of the primary packaging and as a label. The electronic portion 12 comprises processing logic 14, non-volatile memory 15 (or “storage”), an analogue-to-digital converter (ADC) 16, an antenna 17 for near-field communication (NFC) or other form of short-range wireless communication, an energy-harvesting circuit 18 for extracting energy from, for example, the antenna 17 when it is energised and which can be used to provide power to other parts of the circuitry 12, and an NFC or wireless communication protocol engine 19 for suitably formatting data and/or modulating a signal for transmission by the antenna 17 and suitably converting and/or demodulating a signal received by the antenna 17 into data packets. The processing logic 14, non-volatile memory 15, ADC 16, energy-harvesting circuit 18 and protocol engine 19 are implemented in an integrated circuit 20, preferably a flexible integrated circuit, i.e., an integrated circuit printed on a flexible substrate. The ADC 16 may take the form of a 5-bit ADC.

The testing device 2 is battery-less, deriving its power from the energy-harvesting circuit 18. In some cases, the testing device 2 may include a short-term energy storing-capacitor (not shown) for storing harvested power. For example, the capacitor (not shown) may be charged for a given period of time, for example, between 0.5 and 5 minutes or more (for instance, between the start of the test and read time) and the energy stored in the capacitor (not shown) can be used by the device 2 for providing power during measurement, processing and/or transmission. In other cases, the testing device 2 may include a long-term energy storing-capacitor (not shown) for example, in the form of a graphene supercapacitor, which is charged at time of manufacture, and the energy in the capacitor can be used by the device 2 for providing power during measurement, processing and/or transmission.

The interface device 3 takes the form of a smart phone, tablet or other similar type of computing device capable of wireless communication. The interface device 3 comprises a battery 21, a processor-based controller 22, memory 23, an NFC or other form of short-range wireless communication module 24, wireless network interface(s) 25 for example, for communicating via a wireless mobile link, a wireless LAN link, BlueTooth® and/or other similar wireless communication networks, a display 26, input device 27 such as a touch screen, microphone (for voice control), button and/or sliders, one or more cameras 28. When the testing system 1 is used, the controller 22 loads and run software 29 (or “application” or “app”) for controlling transmission of data and power to the testing device 2 and processing received data from the testing device 2.

The system 1 can provide a low-cost diagnostic testing system which takes advantage of using a specialised, but cheap, single-use testing device 2 with a powerful, ubiquitous general-purpose interface device 3. Furthermore, the testing device 2 does not need a battery. Instead, the testing device 2 device can obtain power from the interface device 3 which is used to interrogate the testing device 2. As will be explained in more detail, the testing device 2 is simple and cheap to manufacture thereby resulting in consumable part which can be manufactured in large volumes.

The testing device 2 is generally flat and rectangular and about the size of a credit or playing card. The device 2 can generally take one of two forms depending the nature of the sample to be tested, in particular, the available volume of fluid, its viscosity and the degree to which the fluid needs to be conditioned. In one form, the testing device 2 has a well into which a sample is placed, and this type of device 2 can be used, for instance, to test a sample of blood or oral fluid. In another form, the testing device 2 has a tab (or “wick”) which can be issued (i.e., slide out) and then dipped into the sample, and this type of device 2 can be used with, for instance, urine.

### Well-Based Testing Device 2W

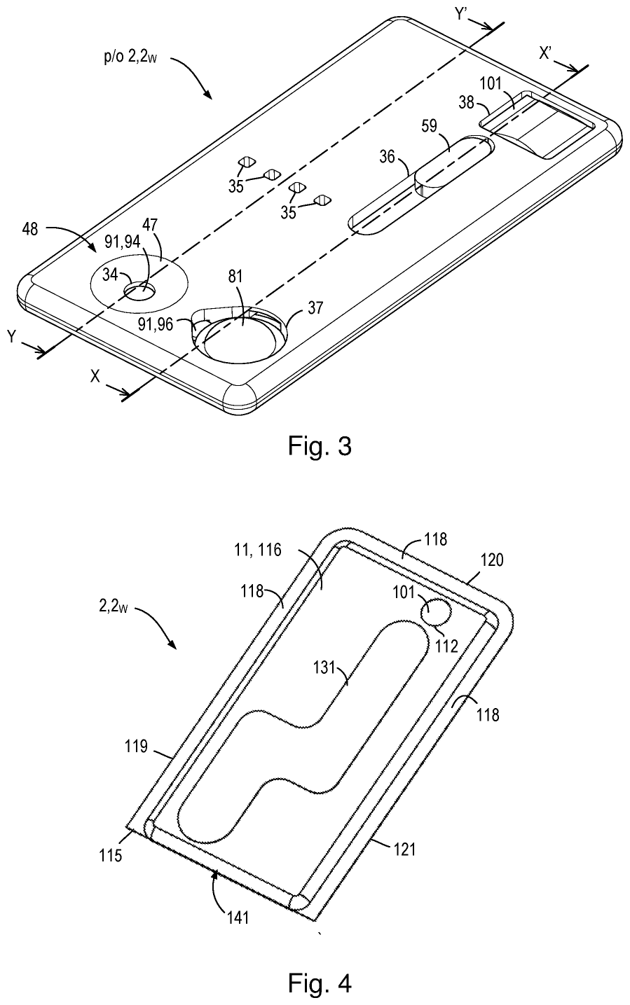

Referring to FIGS. 3 to 6, a well-based testing device 2W is shown.

Referring in particular to FIG. 5, the device 2W takes the form of a multi-piece assembly comprising a generally-flat, rectangular frame 31 (or “housing” or “plate”), a chassis 51 (or “carriage”) which is received in and can slide relative to the frame 31, a set of lateral flow immunoassay strips 9 which sit in the chassis 51 and which are sandwiched between the frame 31 and the chassis 51, a buffer capsule 81 containing buffer 7, a fluid transfer pad 91, and a colour-changing desiccant indicator 101 which are carried by the frame 31, a sheet 111 which enfolds the frame 31 and the chassis 51, and a peel-off sticker 131.

The frame 31 has a first face 32 (or “upper face”) and a second, opposite face 33 (or “lower face”) and first, second and third apertures 34, 35, 36, and first and second recesses 37, 38 (or “blind holes”). The second face 33 is stepped (i.e., multi-level) to form a shallow generally rectangular depression 40 (FIG. 6) surrounded by a raised region 41 (or “ledge”) running around the periphery 42 of the plate 31.

The first recess 37 provides a shallow circular chamber having a side wall 43 with first and second circumferential slots 44, 45 and a floor 46. The second recess 38 provides a shallow rectangular chamber.

The first face 32 of the frame 31 includes a frusto-concial surface 47 which surrounds the first aperture 34 and which has a shallow gradient (e.g., around 15°) so as to form a funnel feeding into first aperture 34. The aperture 34 and funnel 47 is also referred to herein as a well 48 (or “port”).

The chassis 51 has a first portion 52 comprising a generally rectangular frame 53 having an array of one or more elongate recessed channels 54 (or “grooves”), separated by raised ridges 55, each having a rectangular window 56 along a section of the channel 54, a second portion 57 running along one side of the first portion 52. The second portion 57 comprises a generally flat, rectangular wing 58 having a first raised, stadium-shaped member 59 (herein also referred to as a “test actuation slider”, “slider button” or simply “slider”) and a second raised, stadium-shaped member 60 which projects beyond one end 61 of the wing 58 and so provides a spatulate projecting member 62. The first member 59 sits in the third, elongate aperture 36 and serves as a thumb- or finger-actuated slider for slidably moving the chassis 51 within the housing 31. When the chassis 51 is moved, the spatulate projecting member 62 passes through the second slot 45 in the side wall 43 of the chamber 37.

The frame 31 and chassis 51 are made from a suitable plastic material which is preferably biodegradable, such as, polylactic acid (PLA), polybutylene succinate (PBS) or thermoplastic starch (TPS), although polystyrene (PS) or polypropylene (PP) can be used, and which can be formed by injection moulding.

The lateral flow immunoassay strips 9 sit in the channels 54 of the chassis 51 and run over the windows 56. As will be explained in more detail later, when the light sources 10 (FIG. 2) generate light, the light passes through the immunoassay strips 9, through the windows 56 to reach the light or image sensors 11 (FIG. 2). A light sensor can take the form of photodiode. An image sensor can take the form of a flexible image sensor, for example, available from Isorg (www.isorg.fr).

The immunoassay strips 9 each have first and second ends 62, 63 between which are disposed, in order from the first end 62 to the second end 63, a conjugate pad (not shown), a nitrocellulose-based transport membrane (not shown) which supports one or more test lines (not shown) and optional control line (not shown), and a wicking pad (not shown). For transmissive-based measurements, a backing sheet (not shown) supporting is transparent. For reflectance-based measurements, a backing sheet preferably has a white or reflective surface to aid reflection of light.

The buffer capsule 81 takes the form of a pressure-burstable, blister-style pocket formed by a plastic dome 82 and a lidding seal 83 bonded to the edge of the dome 82. The buffer capsule 81 sits in the chamber 41 and bursts when the projecting member 62 enters the chamber 41 through the slot 43. Preferably, the buffer capsule 81 is directional, i.e., bursts in pre-defined direction, and releases its content toward the transfer pad 91.

The transfer pad 91 has a bent funnel shape with a wide end 92, a tapered portion 93 which runs from the wide end to an elbow 94 in a first direction and from which a short strip 95 runs to a narrow end 96 (or “tail”) in a second, perpendicular direction. The transfer pad 91 is non-woven (or “fibrous”) and is formed from a suitable material such as polyethylene. The transfer pad 91 may be secured to the frame by heat staking (or “heat welding”).

Referring to FIGS. 7 and 8, the label 111 is generally opaque but includes a transparent window 112, a first aperture 113 which is larger than the first aperture 34 in the housing 31, and a second elongated aperture 114 having approximately the same dimensions as the third aperture 36 in the housing 31.

The label 111 is folded along a fold line 115 forming two parts 116, 117 (or “wings”). The frame 31, chassis 51, and other parts of the device 2W are assembled and are sandwiched between the two parts 116, 117 of the label 111 such that the transparent window 112 sits over the desiccant indicator 101, the first aperture 113 is aligned (e.g., concentric) with the first aperture 34 in the housing 31 and the second aperture 114 is aligned with the third aperture 36 in the housing 31. The first and second apertures 113, 114 are sealed using the peel-off sticker 131.

The two parts 116, 117 of the label 111 are bonded together, e.g., heat-sealed, along a peripheral region 118 running along the three open sides 119, 120, 121 (FIG. 4) of the folded label 111. Together with the peel-off sticker 131 (FIG. 4), the label 111 provides a gas-tight enclosure and so can serve as primary packaging. Thus, no additional packaging, such as a foil pouch, is required.

The label 111 is provided by the same flexible substrate 13 which carries the circuitry 12. Two substrates, however, may be used and may be laminated together. Thus, a first, inner substrate 13 may carry the circuitry 12 and a second, outer substrate (not shown) may have indicia (such as test name, name of manufacturer, logos, instructions, lot number, expiry date, and the like) as well as carry the peel-off sticker 131.

The flexible substrate 13 is made from a suitable plastic material, such as polyethylene (PE) or polyethylene terephthalate (PET).

The substrate 13 has a face 122 on which conductive tracks 123 made of foil or conductive ink, are formed. The conductive tracks 123 define the antenna 17 and provide contact terminals to the LEDs 10 and photodiodes 11, and the integrated circuit 20. The LEDs 10, photodiodes 11 (or image sensor), and the integrated circuit 20 may, however, be printed.

The desiccant indicator 101 is used to identify whether the test device 2W is good to use or not. For example, a silica gel may be used which is green when dry, but orange when exposed to moisture.

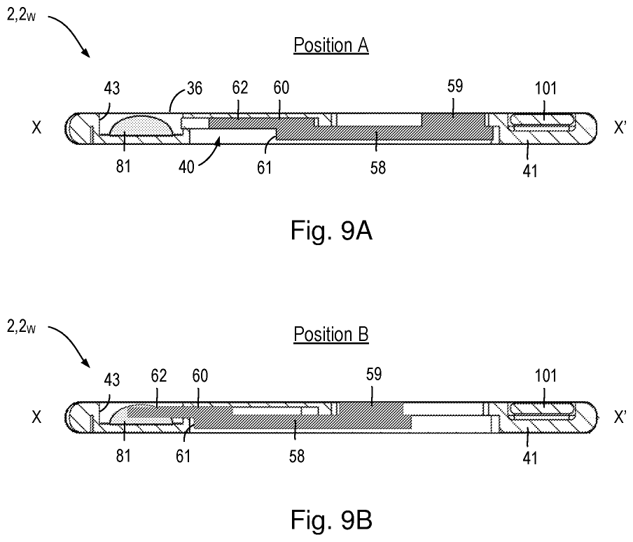

Referring to FIGS. 9A, 10A, and 11A, in a first state, the chassis 51 sits in a first position in the frame 31 of the device 2W. Assuming that a user holds the testing device 2W with a first edge 141 (“lower edge”) closest to them and a second opposite edge 142 (“upper edge”) furthest away, in the first position, the chassis 51 is generally in an upper position. In the position, the slider 59 is in a first position (herein referred to as the “upper position” or “position A”). When the chassis 51 is in a first position, the ends 62 of the lateral flow immunoassay strips 9 are separated from (i.e., not in contact with) the sample and buffer transfer pad 91. In this state, the peel-off sticker 131 is removed to expose the port 48. A sample is added to the port 34 and is deposited on the elbow 94 of transfer pad 91.

Referring to FIGS. 9B, 10B, and 11B, the user can draw (or “slide” of “pull”) the slider 59 to a second position (herein referred to as the “lower position” or “position B”) so that the chassis 51 sits in a second position (“lower position”) in a second state. When the chassis 51 is in the second position, the ends 62 of the lateral flow immunoassay strips 9 overlap and are in contact with the wide end 92 of the sample and buffer transfer pad 91. Furthermore, the act of drawing the slider 59 down causes the projecting member 62 to squeeze the buffer capsule 81, cause it to burst and release the buffer 7 onto the tail 96 of the transfer pad 91. The buffer acts as a chase buffer causing the sample to run onto the lateral flow strips 9.

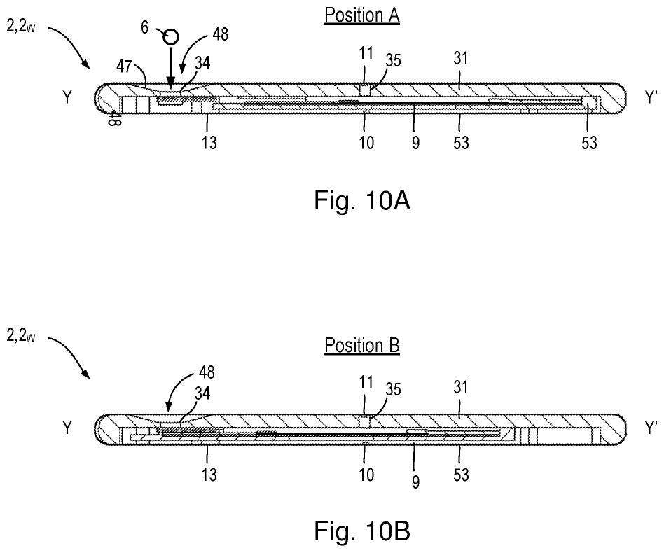

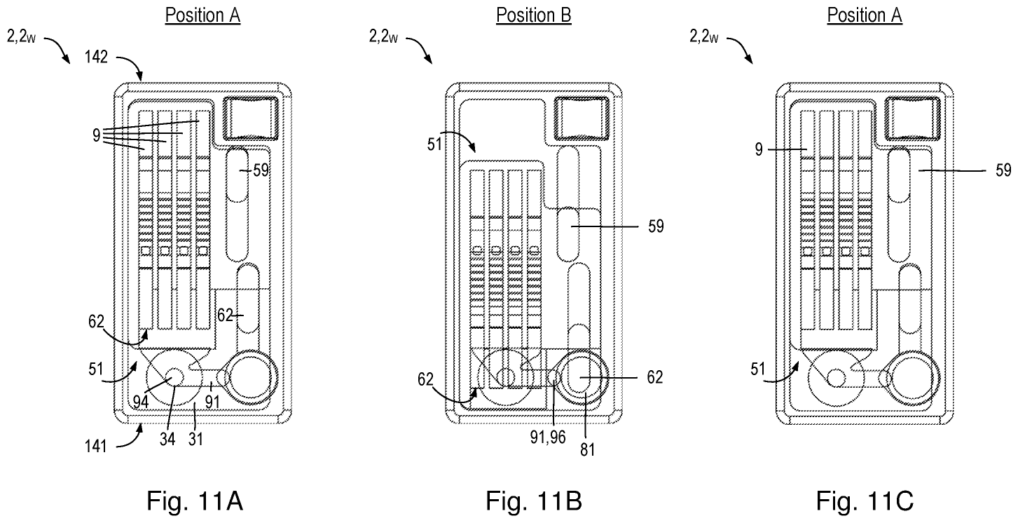

Referring to FIG. 11C, once the test has run and when instructed by an application (which has a timer and which waits an appropriate time according to the test), the user can draw (or “slide” or “push”) the slider 59 back to the first position so that the chassis 51 sits again in the first position. This moves the lateral flow strips 9 between the LEDs 10 (FIG. 10A) and the sensor 11 (FIG. 10A) thereby reading the test.

### Tab-Based Testing Device 2T

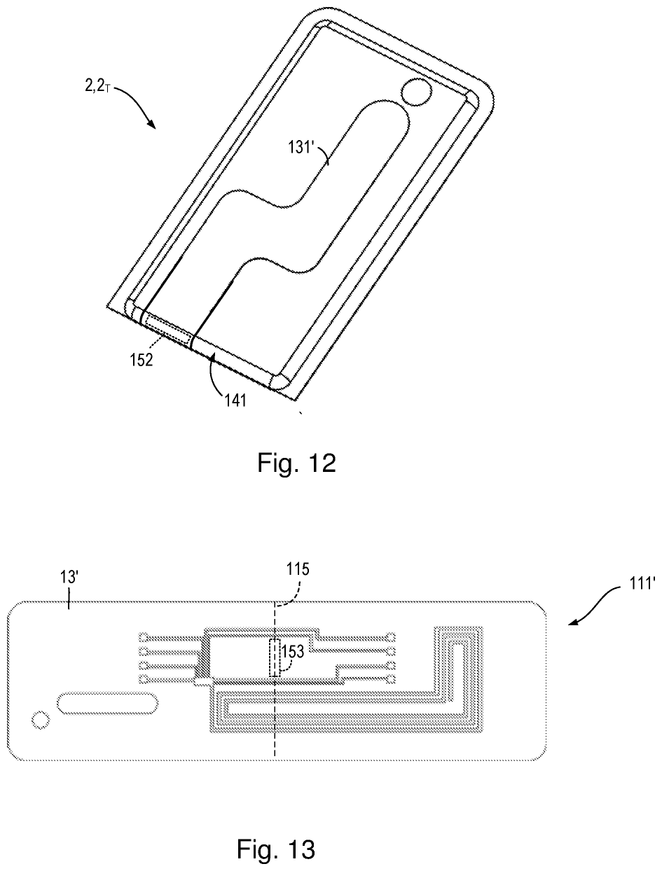

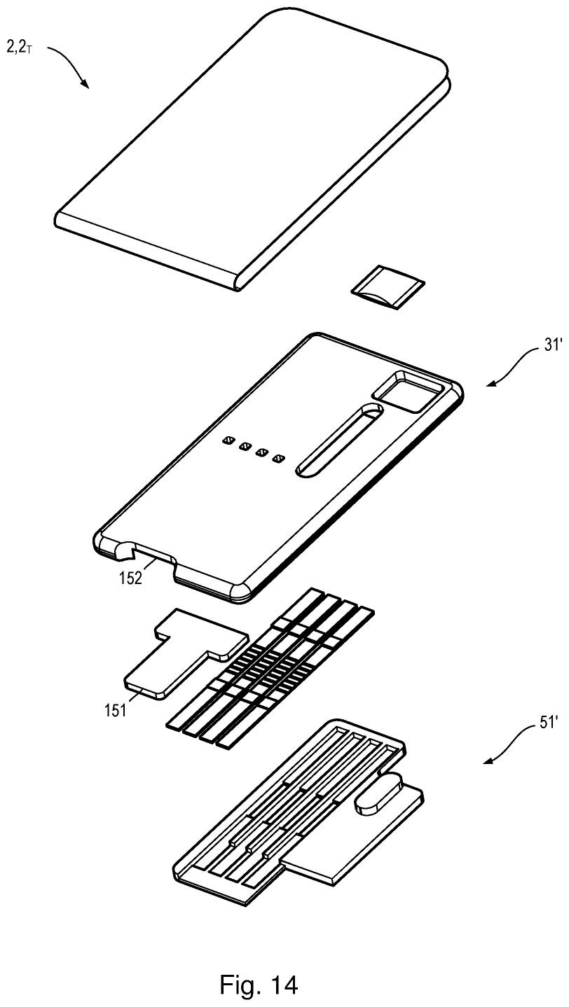

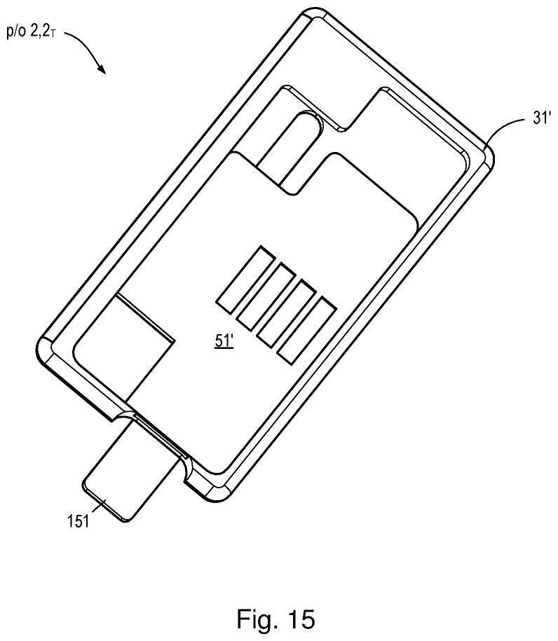

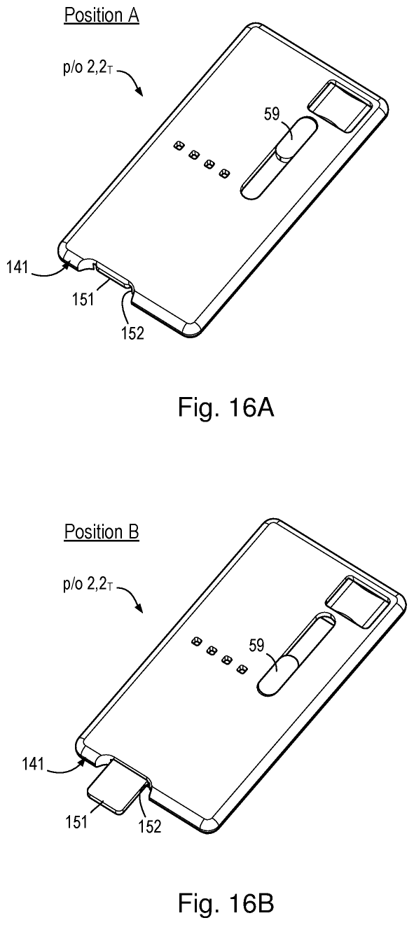

Referring to FIGS. 12, 13, 14, 15, 16A and 16B, a tab-based testing device 2T is shown.

The tab-based testing device 2T is similar to the well-based testing device 2W (FIG. 4) but differs generally in that it does not use a port 34 (FIG. 3) to receive a sample 6 or employ a buffer 7 (FIG. 5), but instead uses an elongate sample transfer pad 151 (or “wick element”) which is dipped in the sample. The sample transfer pad 151 is connected to and is in direct contact with the lateral flow strips 9 so that the sample 6 is transferred onto the lateral flow strips 9. The sample transfer pad 151 moves with the chassis 51 such that it can travel from a retracted position, housed in the device 2, emerge through a slot 152 in the lower edge 141 of the device 2, to a deployed position which allows the transfer pad 151 to be dipped in the sample 6.

Thus, in the tab-based testing device 2T, the main housing 31′ does not include a first aperture 34 (FIG. 5) and the chamber 37 (FIG. 5), the chassis 51′ does not include a second member 60 (FIG. 5) and spatulate projecting member 62 (FIG. 5), the label 111′ does not include aperture 113 (FIG. 5) and the device does not have a buffer capsule 81 (FIG. 5) or the sample and buffer transfer pad 91 (FIG. 5). Instead, the testing device 2T has a generally rectangular transfer pad 151 and a slot 152 in the lower edge 141 of the device 2T. The label 111′ includes a slot 153 along the fold line 115, and the peel-off sticker 131′ is made longer to reach the opposite face of the device and so cover the slot 152.

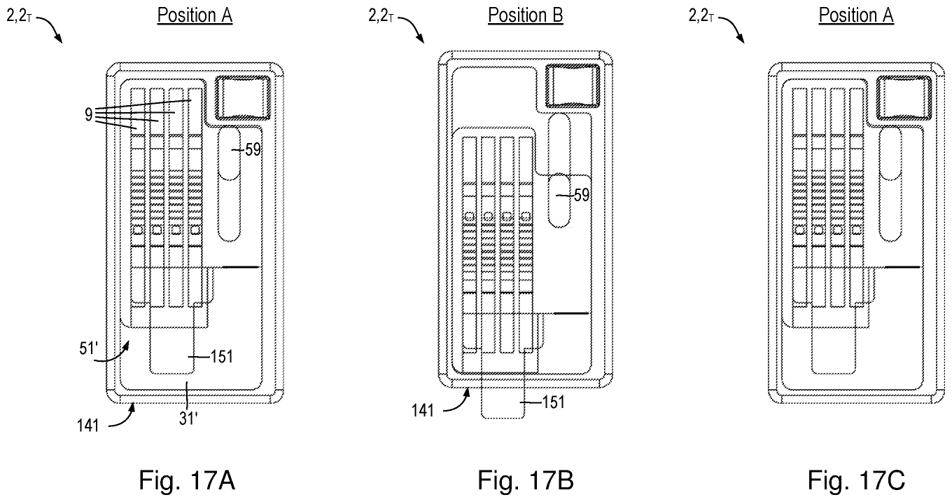

Referring to FIGS. 17A to 17C, the tab-based testing device 2T can be moved between the first and second positions in the same way as the well-based testing device 2W (FIG. 5) described earlier using the slider 59.

### Variants of Testing Device 2

In the devices 2W (FIG. 4), 2T (FIG. 11) hereinbefore described, there are four lateral flow strips 9, each provided with a light source-light/image sensor pair 10, 11.

A device (well-type or tab-based testing device) can include fewer or more lateral flow strips 9. Moreover, each lateral flow strip 9 may include different arrangements of light sources 10 and light/image sensors 11, e.g., to perform a reflectance measurement. Furthermore, the number of light sources 10 need not be the same as the number of light/image sensors 11. For example, more than one light source 10 may share one light/image sensors 11 and/or more than one lateral flow strip 9 may share the same light sources 10 or light/image sensors 11. Different light sources 10 can emit light at different wavelengths.

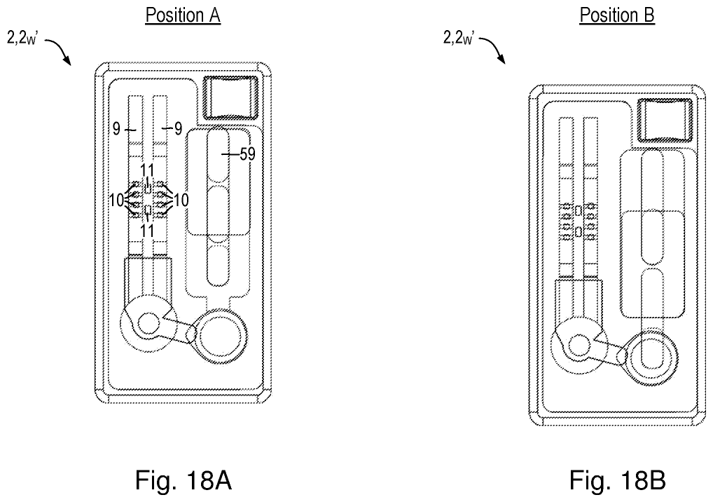

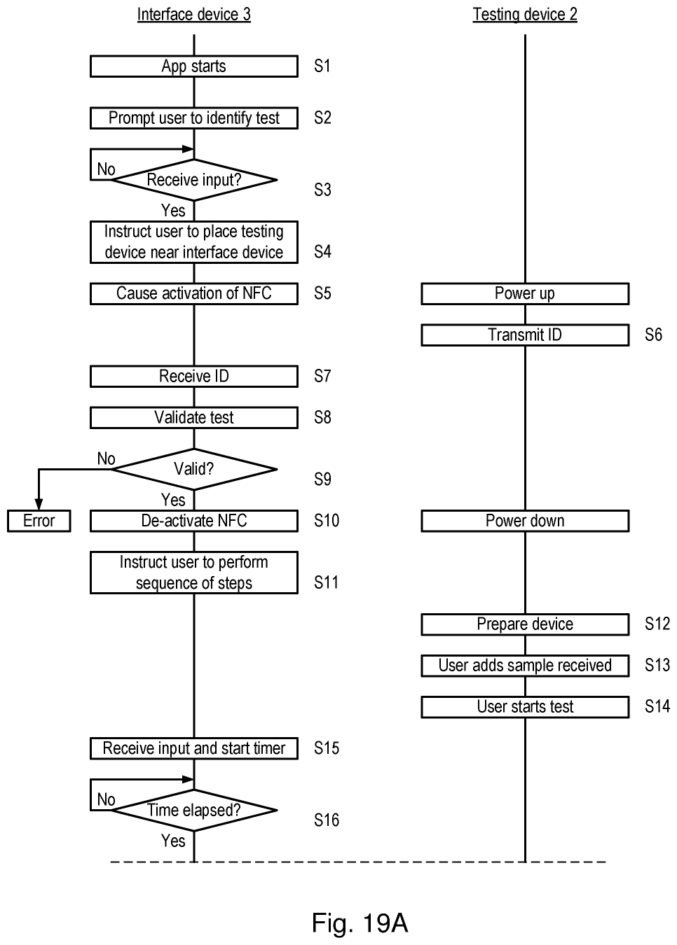

Referring to FIGS. 18A and 18B, a variant of a well-based testing device 2W′ is shown.

The well-based testing device 2W′ is similar to the well-based testing device 2W (FIG. 5) hereinbefore described except that there are only two lateral flow strips 9 and each lateral flow strip 9 is provided, along their length, with more than one light source 10 (in this case, four light sources 10 which in this case are in the form of LEDs), one or more light/image sensors 11 (in this case, light/image sensors 11 which in this case are in the form of photo diodes) are positioned between the lateral flow strips 9 and the light sources 10 and light/image sensor(s) 11 are arranged on the same side of the lateral flow strip 9 (in this case above the lateral flow strips 9) so as to perform a reflectance measurement.

Thus, the variant of a well-based testing device 2W′ can provide a multiplexed test, using only two lateral flow strips 9. The device 2W′ can provide tests for six analytes, three per strip, plus one control line per strip. The device 2W′ can be arranged for static read, namely, the position of the light sources 10 and light image sensors 11 are fixed relative to the line positions on the lateral flow strips.

### Operation

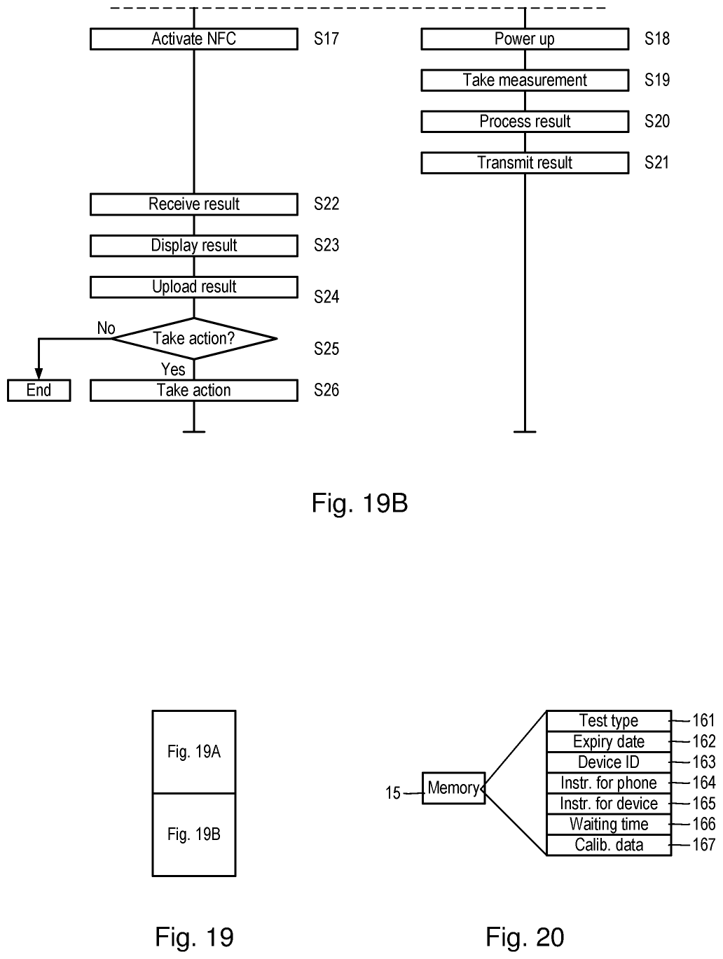

Referring to FIGS. 1, 19 and 20, operation of the testing system 1 will now be described.

When a user, who may be the subject of the test or another person, such as a doctor, nurse, or a law-enforcement officer, is ready to perform a test, he or she opens the application 29 on their interface device 3.

The controller 22 loads and runs the application 29 (step S1) and prompts the user, via the display 26 and/or by another means, such as voice notification, to identify the test (step S2). In some cases, this may be done by using one or more pull-down menus.

The application 29 instructs the user to check the colour of the colour-changing desiccant indicator 101 (FIG. 5) through the transparent window 112. If the desiccant indicator 101 is the correct colour, then the user can proceed to the next step. If, however, the desiccant indicator 101 is the incorrect colour (which may occur if the enclosure provided by the label 111 and sticker 131 has been opened or damaged), then the user is instructed not to use the test device 2 and, instead, to discard it.

Once the controller 22 has received the identity of the type of test from the user (step S3), the controller 22 instructs the user, via the display 26 and/or via another means, to bring the testing device 2 and the interface device 3 close together (step S4), which automatically activates the NFC module 24 (step S5). If the testing device 2 and the interface device 3 are sufficiently close, then the energy harvesting module 18 is able to harvest energy to energise the processing logic 14, and the processing logic 14 transmits information about the test type 161 and expiry date 162 held in non-volatile memory 15 (step S6). The processing logic 14 may transmit other information 163, 164, 165 including instructions 164 for the application, including information to be presented to the user and how to process user inputs, a waiting time 166 and/or calibration data 167.

The controller 22 receives the information, via the NFC module 24, and checks the validity of the test (steps S7 & S8). Checking the validity of the test may include the interface device 3 checking information received from the testing device 2 which is stored in non-volatile memory 15. Additionally, or alternatively, checking the validity of the test may include the interface device 3 interrogating a remote server (not shown) which provides access to a database (not shown) of tests. If the test is invalid, then the controller 22 informs the user via the display 26 (step S9). The controller 22 de-activates the NFC module 24 (step S10) resulting in power-down of the testing device 3 (“entering sleep mode”).

The controller 22 instructs the user to prepare the testing device 3 and to perform a test (step S1). This may be performed by presenting a series of instructions on screen (i.e., via display 26) or even through spoken instructions via a speaker (not shown).

The controller 22 instructs user to prepare the device 2 including removing the sticker and reading the device 2 to receive a sample (step S12), for example, by moving the slider 59 (FIG. 10A; FIG. 13A) from position A (FIG. 10A; FIG. 13A) to position B (FIG. 10A; FIG. 13B). Depending on the type of test, the controller 22 instructs the user when to take a sample.

The user adds the sample to the device 2 (step S13), for example, via a port or using the wick element, and, if necessary, starts the test (step S14).

The controller 22 prompts the user to press “Start” on the application and, once the user has pressed “Start”, the interface device 3 starts a timer, e.g., a countdown timer (step S15). Preferably, the protocol sequence for running the test and the required waiting time are received from the testing device 2 which are stored in non-volatile memory 15.

Once a predetermined period of time has passed (step S16), the controller 22 activates the NFC module 24 (step S17) and the energy harvesting module 18 is able to harvest energy to energise the processing logic 14 and other parts of the circuit 12 (step S18).

The processing logic 14 takes a measurement (step S19), for example, by activating light sources 10 (FIG. 2), if necessary, in a suitable order and receiving measurements from the light/image sensor 11. For example, this may comprise reading each line (not shown) on each strip sequentially. The processing logic 14 may process the measurements to obtain a result (for example, “positive” or “negative”) (step S20) and transmit the measurement and/or a result (step S21). The non-volatile memory 15 can store device-specific information 167 which is embedded in memory 15 during manufacture. The device-specific information 167 may include, for instance, assay calibration data 167 and instructions on how the processing logic 14 should take the measurement and generate a result from it.

The controller 22 receives the measurement and/or a result, via the NFC module 24 (step S22) and may display the measurement and/or result (Step S23). Additionally, or alternatively, the controller 22 may store and/or upload the measurement and/or result to a remote server (step S24).

Depending on the type of test or use case, the controller 22 may take further action (step S26). Action may be, for example, instructing the user to reapply the peel-off sticker 131 so as to re-seal the device and to send the testing device to a laboratory to confirm the result or for further testing.

### Use Cases

The testing system 1 can be used in a number of different scenarios.

Referring to FIG. 21, the testing system 1 can be used by a subject for performing a test at home, work or other user location 201. The user (not shown) can perform the test and upload the measurement and/or results to a remote server 202 for example, at a patient record repository, laboratory information system, hospital information system or other similar location or authority 203. Uploading the measurement and/or result may happen automatically, i.e., without user instruction. A clinician (not shown) may access the measurement and/or results using their computer 204 located at a hospital, surgery, clinic or other similar type of location 205.

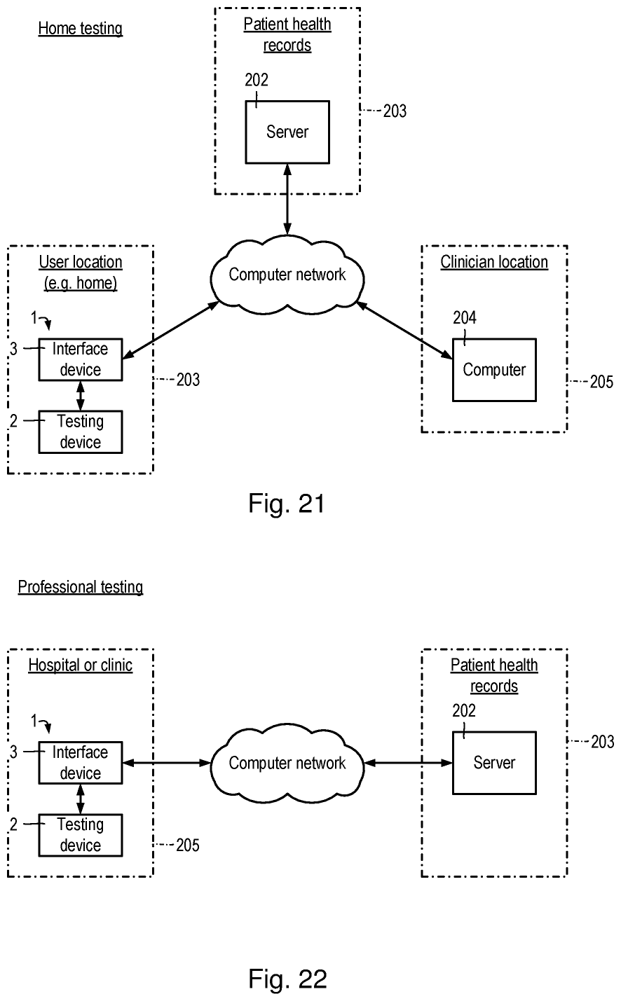

Referring to FIG. 22, the testing system 1 can be used by clinician (not shown) at a hospital, surgery, clinic or other similar type of location 205. The user (not shown) can perform the test and upload the measurement and/or results to a remote server 202 for example, at a patient record repository, laboratory information system, hospital information system or other similar location or authority 203.

### Alternative Fluid Processing and Measuring Sections

Referring again to FIG. 1, as explained earlier, the fluid processing section 4 can take other forms. For example, the fluid processing section 4 may take the form of a microfluidic system in which the sample and reagent(s) and optional buffers are processed, e.g., metered, mixed and allowed to react. Reactions and reaction products can be measured optically for example using light sources 10 and/or light/image sensors 11 or using other remote processes, i.e., processes which do not necessarily involve a sensor 11 coming into direct contact with fluids (unlike ones which involve direct contact with the fluid, e.g., using an electrode which is wetted by the fluid).

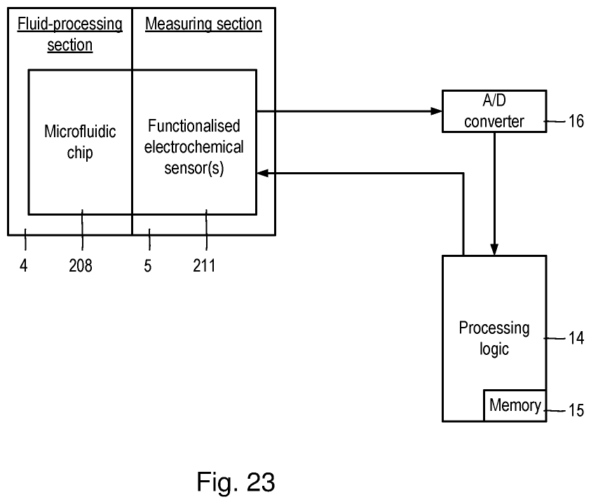

Referring to FIG. 23, in another arrangement, a fluid-processing section 4 may include a microfluidic front-end 208 (or “chip”) and a measuring section 5 which consists of one or more direct-contact sensors 211, such as functionalised electrochemical sensors. A functionalised electrochemical sensor may take the form of an electrode (not shown) or wire (not shown) coated with a receptor (not shown) which binds to a specific target molecule (not shown) and changes in electrical properties (such as current and/or resistance) are measured. The electrode (not shown) may take the form of a gold, carbon or graphene pad printed on the substrate 13 and coated with a receptor.

### Modifications

It will be appreciated that various modifications may be made to the embodiments hereinbefore described. Such modifications may involve equivalent and other features which are already known in the design, manufacture and use of assay devices and component parts thereof and which may be used instead of or in addition to features already described herein. Features of one embodiment may be replaced or supplemented by features of another embodiment.

Measurements need not be performed while the chassis is stationary (i.e., static). Measurement can be performed while the chassis is moving (i.e., kinetic). Thus, lines in a lateral flow strip may move relative to the light source and light/image detector.

In some cases, the test area (such as a microfluidic part) may be held in or by the frame, i.e., not by the chassis.

Although claims have been formulated in this application to particular combinations of features, it should be understood that the scope of the disclosure of the present invention also includes any novel features or any novel combination of features disclosed herein either explicitly or implicitly or any generalization thereof, whether or not it relates to the same invention as presently claimed in any claim and whether or not it mitigates any or all of the same technical problems as does the present invention. The applicants hereby give notice that new claims may be formulated to such features and/or combinations of such features during the prosecution of the present application or of any further application derived therefrom.

1-26. (canceled)

27. A wrapper for a diagnostic test device, the wrapper comprising:
a flexible substrate; and
an electronic section supported on the flexible substrate, comprising:
a measurement section for measuring properties in or of a test area;
an antenna;
a modulator-demodulator coupled to the antenna;
a data processing unit coupled to the measurement section and to the modulator-demodulator; and
an energy-harvesting unit coupled to the antenna and to the data processing unit;
wherein the data processing unit is configured, upon receiving a measurement from the measurement section, to generate a message containing the measurement and/or a result of processing the measurement and to transmit the message via the modulator-demodulator and the antenna.

28. The wrapper of claim 27, wherein the measurement section comprises:
at least one light source; and
at least one light/image sensor;
wherein the at least one light source and the at least one light/image sensor are arranged to measure transmission, absorbance, and/or reflectance through or by the test area.

29. The wrapper of claim 28, wherein each test area is provided with at least one pair comprising a light source and a light/image sensor.

30. The wrapper of claim 27, wherein the measurement section comprises:
an electrochemical sensor.

31. The wrapper of claim 30, wherein the electrochemical sensor comprises an electrode or wire coated with a receptor which binds to a specific target molecule.

32. A diagnostic test device, comprising:
a sample-processing section comprising:
a test area containing a test reagent; and
a fluid conveyor for receiving a liquid sample and transferring the liquid sample to the test area;
a frame;
a chassis coupled to the frame and slidably moveable with respect to the frame, using a slider, between first and second positions, wherein the chassis may carry the test area and wherein, in the second position, the sample is transferable to the test area or the sample is receivable by the fluid conveyor;
the wrapper of claim 27, wherein the wrapper encloses the frame and chassis, and wherein the wrapper includes a first aperture for allowing the fluid conveyor to receive the sample and a second aperture for allowing a user to access to the slider; and
at least one removeable sticker arranged to cover the first and second apertures.

33. The diagnostic test device of claim 32, comprising:
at least one lateral flow strip, each strip providing a respective test area.

34. The diagnostic test device of claim 33, comprising:
two, three or four lateral flow strips.

35. The diagnostic test device of claim 32, wherein the first aperture is aligned with the fluid conveyor such that, in the first position, the sample is receivable by the fluid conveyor and, in the second position, the test area is in fluid communication with the fluid conveyor.

36. The diagnostic test device of claim 32, further comprising:
a burstable buffer capsule arranged such that moving chassis between the first and second positions cause the burstable buffer capsule to burst and release the buffer onto conveyor pad.

37. The diagnostic test device of claim 36, wherein the burstable buffer capsule is held in chamber the frame and wherein the chassis comprises a projecting member arranged to enter into the chamber when the chassis is moved to into the second position.

38. The diagnostic test device of claim 32, wherein the chassis carries the fluid conveyor and the test area is in permanent fluid communication with the fluid conveyor, such that, in the first position, the fluid conveyor is stowed inside the wrapper, and, in the second position, the fluid conveyor is deployed such that the sample is receivable by the fluid conveyor.

39. The diagnostic test device of claim 32, wherein the wrapper and the at least one removeable sticker are arranged to provide a gas-tight enclosure.

40. The diagnostic test device of claim 32, further comprising:
a colour-changing desiccant indicator;
wherein the wrapper includes a transparent window positioned such that the colour-changing desiccant indicator is visible.

41. The diagnostic test device of claim 32, which is battery-less.

42. An interface device for providing an interface to a diagnostic test device, the device comprising:
a controller,
a short-range wireless communication module,
a user interface; and
a user input device
wherein the controller is configured:
to provide instructions to a user, via the user interface, to perform a test using the diagnostic test device;
to receive an input from the user, via the user input device, to start a timer;
to determine if a given period of time has passed;
upon a positive determination, to activate the short-range wireless communication module so as to provide power to the diagnostic test device;
to receive a signal from the diagnostic test device; and
to present the result to the user, via the user interface.

43. A diagnostic test system comprising:
the diagnostic test device of claim 32; and
the interface device for providing an interface to a diagnostic test device, the device comprising:
a controller,
a short-range wireless communication module,
a user interface; and
a user input device wherein the controller is configured:
to provide instructions to a user, via the user interface, to perform a test using the diagnostic test device;
to receive an input from the user, via the user input device, to start a timer;
to determine if a given period of time has passed;
upon a positive determination, to activate the short-range wireless communication module so as to provide power to the diagnostic test device;
to receive a signal from the diagnostic test device; and
to present the result to the user, via the user interface.

44. A diagnostic test system comprising:
the diagnostic test device of claim 32; and
a hand-held communications device capable of short-range wireless communication with the diagnostic test device.

45. A method, comprising:
upon receiving power from an energy-harvesting module a first time:
transmitting data stored in non-volatile memory via a short-range communication module;
upon receiving power from the energy-harvesting module a second, later time:
retrieving instructions from the non-volatile memory;
performing a measurement using a measuring section and processing measurement data from the measuring section in dependence upon the instructions; and
transmitting measurement data and/or a result obtained by processing the measurement data via the short-range communication module.

46. The method of claim 45, wherein the data comprises a test type and/or an expiry data.

47. The method of claim 45, wherein the data comprises instructions to be presented to a user.

48. The method of claim 45, wherein the data comprises calibration data.

49. Logic arranged to perform the method of claim 45.

50. A method, comprising:
causing transmission of a short-range wireless communication signal to a testing device for providing the testing device with power;
receiving data wirelessly from the testing device;
providing instructions to a user, via a user interface, to perform a test using the diagnostic test device in dependence upon the data received from the testing device;
receiving an input from the user, via a user input device, to start a timer;
determining if a given period of time has passed;
upon a positive determination, causing transmission of a further short-range wireless communication signal to the testing device for providing the testing device with power;
receiving a signal from the diagnostic test device, the signal containing measurement data and/or result; and
to present the measurement and/or result to the user via the user interface.

51. The method of claim 50, wherein the given period is specified in the data received from the testing device.

52. A computer program product comprising a computer-readable medium which is non-transitory storing thereon the computer program comprising instructions for performing the method of claim 50.
